(19) United States
(12) Patent Application Publication
(10) Pub. No.: US 2026/0017720 A1
(43) Pub. Date: Jan. 15, 2026

(54) METHOD OF MINING VIRTUAL CURRENCY BASED ON USER'S BODY REACTION

(71) Applicant: IEUMCOMPANY INC., Seoul (KR)

(72) Inventor: Minjae LEE, Seoul (KR)

(21) Appl. No.: 18/721,187

(22) PCT Filed: Oct. 23, 2023
(86) PCT No.: PCT/KR2023/016514

§ 371 Date: Sep. 2, 2025

(30) Foreign Application Priority Data
Oct. 7, 2023 (KR) 10-2023-0133848

(51) Int. Cl. G06Q 40/06; A61B 3/11; A61B 5/00; A61B 5/0205; A61B 5/024; A61B 5/08; A61B 5/372

(52) U.S. Cl. CPC G06Q 40/06; A61B 5/02055; A61B 3/112; A61B 5/024; A61B 5/0816; A61B 5/372; A61B 5/4812; A61B 2503/12; G06Q 2220/00

(57) ABSTRACT

Provided is a method for varying the amount of virtual currency mining based on a user's body responses. In a virtual currency mining server according to one embodiment, the method includes: acquiring physical activity information from a user terminal; determining the physical state of the user based on the physical activity information; and outputting a signal to mine virtual currency based on the physical state of the user.

### FIELD OF INVENTION

The present invention relates to a method of mining virtual currency based on the user's body reaction.

### BACKGROUND OF INVENTION

Virtual currency (virtual currency or virtual money) refers to digital currency or electronic money that is used in electronic form in a specific virtual space connected by a network without physical objects such as bills or coins. According to the definitions provided by the European Central Bank (ECB), the U.S. Treasury Department, and the European Banking Authority, a cryptocurrency is a type of digital currency that is not regulated by the government, is issued and managed by developers, and can only be used as a means of payment in a specific virtual community.

While most of blockchain-based cryptocurrencies are decentralized in the sense that there is no central point of control, blockchain-based cryptocurrencies can also be implemented in a centralized system having a central point of control over the cryptocurrency. A blockchain is a data structure that stores a list of transactions and corresponds to a distributed electron that records transactions between the source identifier(s) and the target identifier(s). Computer resources (or nodes, etc.) can maintain the blockchain and cryptographically validate each new block and the transactions contained in that block. As the interest in blockchain-based cryptocurrencies has increased, research on how to mine them is also actively ongoing. In mining virtual currency, it may be necessary to efficiently distribute the work in this regard as it consumes a lot of electricity power and loads the device. In addition, there is active research being conducted to adjust the mining weight of virtual currency based on the user's physical activity.

### DETAILED DESCRIPTION OF THE INVENTION

### Technical Problem

The present invention provides a method for varying the amount of cryptocurrency mining based on the user's body response.

### Technical Solution to the Problem

A virtual currency mining server based on the user's physical condition according to one embodiment may include: acquiring physical activity information from a user terminal; determining the user's physical condition based on the physical activity information; and outputting a signal to mine virtual currency based on the user's physical condition.

Physical activity information according to one embodiment may include the user's EEG information, pulse rate information, body temperature information, pupil movement information, and breathing pattern information.

In one embodiment, the virtual currency mining server according to the user's physical state may further include: determining the sleep state based on the user's physical state; and the step of outputting a signal for mining the virtual currency based on the user's physical condition may include: adjusting the amount of virtual currency mined according to the sleep state.

The sleep state of the user according to one embodiment is divided into the first stage of sleep, the second stage of sleep, and the third stage of sleep based on the EEG information, pulse rate information, body temperature information breathing pattern information. The stage of outputting the signal to mine the virtual currency based on the physical state of the user may include: adjusting the amount of virtual currency mined according to the first stage of sleep, the second stage of sleep, and the third stage of sleep.

The stage of classifying the sleep state of the user as the first stage of sleep according to one embodiment may include: classifying the user's sleep state as the first stage of sleep if, based on the user's pupil movement information, the user's pupil moves more than once a predetermined distance for a predetermined first time; if, based on the user's breathing information, the number of the user's breathing rate is less than a predetermined cycle; and if the user's breathing sound exceeds a predetermined decibel.

### Advantageous Effects of the Invention

The present invention can maximize the user's satisfaction by varying the amount of virtual currency mining for the user according to the user's sleep pattern.

The present invention analyzes the user's sleep pattern and thereby mines virtual currency, thereby improving the efficiency of virtual currency mining.

### BRIEF DESCRIPTION OF THE DRAWINGS

The accompanying drawings, which are included as part of the detailed descriptions to facilitate the understanding of the various embodiments, provide various embodiments and, along with detailed explanations, explain the technical characteristics of the various embodiments.

FIG. 1 is a drawing illustrating the operation of the processor by embodiment.

FIG. 2 is a drawing of the operation of the server by embodiment.

FIG. 3 is a flow chart that exemplifies the operation of setting the mining amount of virtual currency differently by one embodiment.

FIG. 4 is a flowchart illustrating the operation of setting the amount of mining differently according to the surface stage according to the embodiment.

FIG. 5 is a drawing illustrating the operation of mining virtual currency according to the user's sleep state by one embodiment.

### EMBODIMENTS OF THE INVENTION

The present invention can be modified in various ways and may have several embodiments, and the specific embodiments are illustrated in the drawings and described in detail in the detailed description. However, this is not intended to limit the present invention to a particular embodiment and should be understood to include all modifications, equivalents, or substitutions that fall within the scope of thought and technology of the invention. In describing each drawing, similar reference marks were used for similar components.

The terms 1, 2, A, B, etc., may be used to describe various components, but the components should not be limited by the above terms. The above terms are used only to distinguish one component from another. For example, without going beyond the scope of the rights of the present invention, the first component may be named the second component and similarly the second component may be named the first component. The term “and/or” includes a combination of multiple related items or any of the multiple related items.

When a component is said to be “connected” or “linked” to another component, it must be understood that it may be directly connected or linked to that other component, but there may be other components in between. On the other hand, when it is stated that a component is “directly connected” or “directly linked” to another component, it should be understood that there is no other component in between.

The terminology used in the present application is used only to describe a particular embodiment and is not intended to be specific to the present invention. Singular expressions include plural expressions unless the context indicates otherwise. In this application, the terms “include” or “have” shall be understood to designate the existence of the features, numbers, steps, operations, components, parts, or combinations thereof described in the specification, and do not preclude the existence or addition of one or more other features or numbers, steps, operations, components, parts, or combinations thereof.

Unless otherwise defined, all terms used herein, including technical or scientific terms, have the same meanings as are generally understood by persons with ordinary knowledge in the technical field to which the invention belongs. Commonly used terms such as those defined in the dictionary should be construed as having meanings consistent with their meanings in the context of the relevant description, and should not be construed in an idealistic or overly formal sense unless clearly defined in this application.

Below, referring to the attached drawings, a preferred embodiment of the present invention will be described in more detail. In describing the present invention, for ease of overall understanding, the same reference code is used for the same component on the drawing, and duplicate descriptions for the same component are omitted.

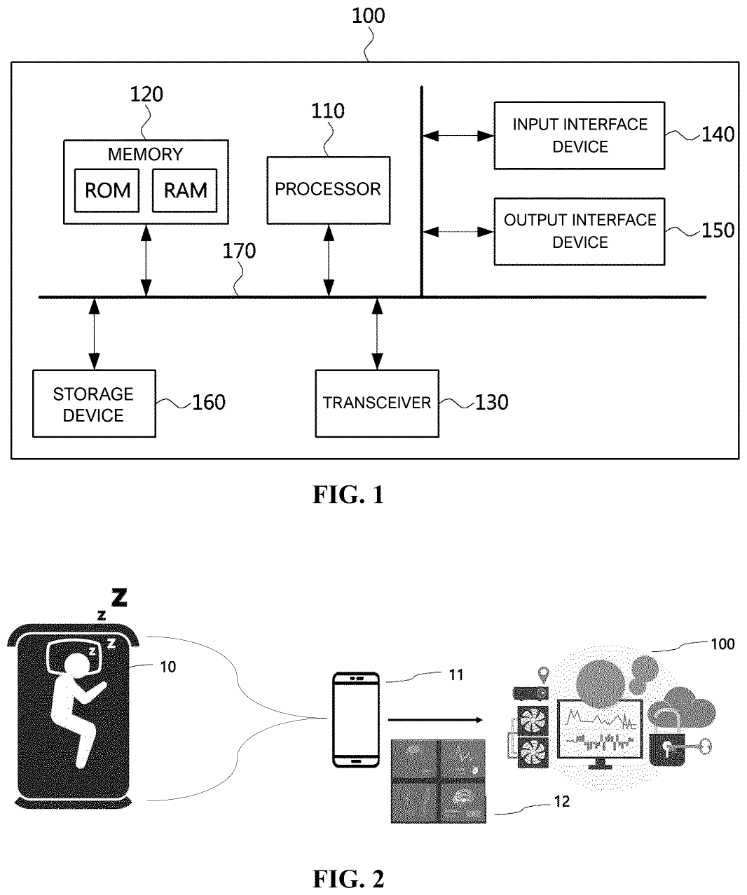

FIG. 1 is a drawing illustrating the operation of a processor belonging to a server (100) by one embodiment. Specifically in FIG. 1, at least one processor (110) is the central processing unit (central processing unit, CPU), graphics processing unit (GPU), or a dedicated processor on which the methods described in the embodiments of the present invention are performed. Memory (120) and storage device (160) may each consist of at least one of volatile and non-volatile storage media. For example, the memory (120) may be either read-only memory (ROM) or random access memory (RAM), and the storage device (160) may be flash memory, a hard disk drive (HDD), a solid-state drive (SSD), or a memory card (e.g., a micro SD card).

In addition, the server (100) may include a transceiver (130) capable of communication via a wireless network, which can be included in the server. In addition, the server (100) may further include an input interface device (140), an output interface device (150), a storage device (160), and more. Each of the components contained in the server (100) is connected by bus (170) so that they can communicate with each other.

FIG. 2 is a drawing of the operation of the server by embodiment.

Specifically in FIG. 2, in the virtual currency mining service according to the physical state of user (10), user (10) can obtain physical activity information from terminal (11), and the physical condition of user (10) can be determined based on the physical activity information, and based on the physical activity information, the virtual currency mining signal can be output based on the physical condition of the user (10). The virtual currency mining server is a server that can mine virtual currency according to the physical condition of the user (10), and mining here refers to the process of adding transaction records to the public ledger of virtual currency, for example, Bitcoin, which is called blockchain, and mining can refer to the act of creating a block that records the transaction history of cryptocurrency and obtaining cryptocurrency in return. This can refer to the process of using computer processing power to keep a record of performance. Here, blockchain can represent a decentralized public ledger where all transactions of virtual currencies such as Bitcoin are recorded. Cryptocurrencies are distributed and stored on a global network of ledgers of transactions without an issuing authority such as a central bank and are designed to provide a certain reward to the person who creates the block to maintain the blockchain. For example, in the case of Bitcoin, one every 10 minutes a new block is created, and the person who finds a total of 64 hashes with the name of the block in hexadecimal digits can be issued and rewarded with bitcoins. At this time, mining cryptocurrency can be carried out directly on the server that can mine the virtual currency, and the signal to mine the virtual currency can be output using an external server. The server can perform the virtual currency mining method according to the user's physical condition.

Physical activity information may include the user's brain wave information, pulse rate information, body temperature information, pupil movement information, and breathing pattern information. The user's EEG information, pulse rate information, body temperature information, pupil movement information, and breathing pattern information may be obtained from the user's (10) terminal (′ or through the information transmitted to the user's (10) terminal after acquiring the user's EEG information using another device. In addition, physical activity information is a wide range of personal information and health information of the user (10), and disease information may be included. In other words, depending on the health status or disease status of the user (10) or the preferred information, it can be set to vary the amount of virtual currency. Mining virtual currency can be done by universal formulas. For example, as a POW (proof-of-work method), data can also be processed through hash processing, which is a data processing method, and mining machines such as graphics cards can be used in this process. In addition, you can use proof-of-stake (POS), which allows you to have a stake in a coin and have the power to create blocks with your stake and be rewarded with interest. In addition, it is possible to mine currency through the user (10) terminal, which is not limited to a specific method, but can be done by various methods, and can be done universally.

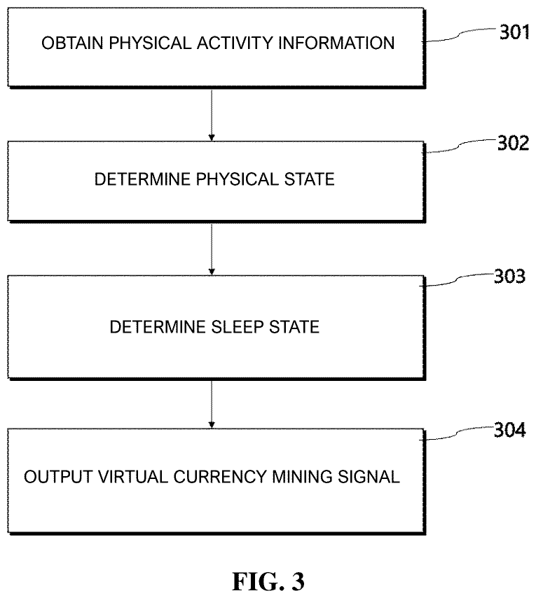

FIG. 3 is a flow chart that exemplifies the operation of setting the mining amount of virtual currency differently by one embodiment.

Specifically in FIG. 3, the server can acquire physical activity information (301), determine the body state (302), determine the sleep state (303), and output the virtual currency mining signal (304). As described in detail later, a signal can be output to vary the amount of virtual currency mining according to the sleep state, and the sleep state can be set differently depending on the amount of sleep, breathing rate, brain wave information, and pupil movement.

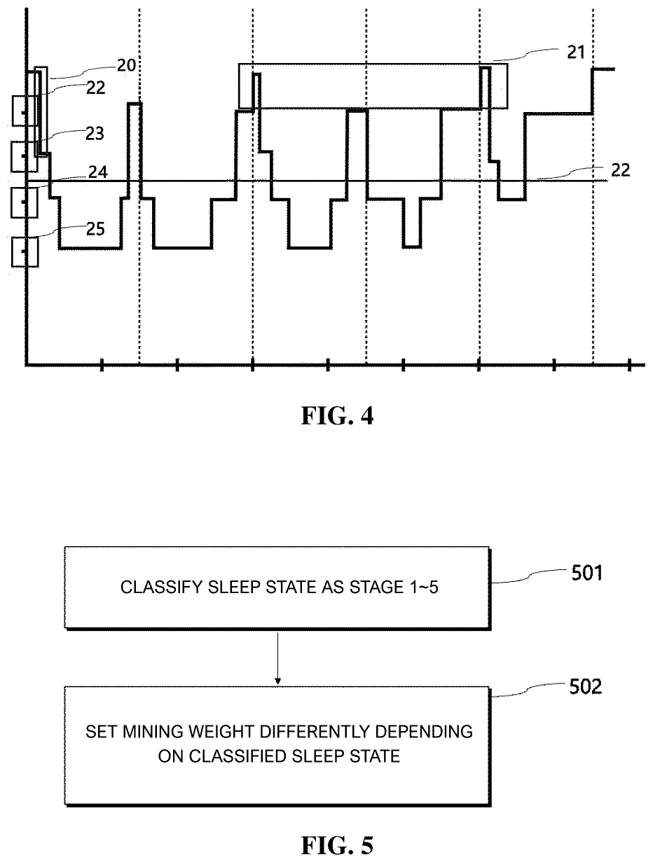

FIG. 4 is a flowchart illustrating the operation of setting the amount of mining differently according to the surface stage according to the embodiment.

Specifically in FIG. 4, the user can fall asleep in Awakening, that is, in the waking state, into rem sleep (the first stage of sleep), and then gradually fall asleep in the second, third, fourth, and fifth stages of sleep. The sleep-wake cycle can be delicately controlled by the body's circadian rhythm, in which homeostatic processes and changes in daylight conditions play an important role. Prolonged wakefulness periods or high loads on the body (caused by large amounts of vigorous physical activity by daily embodiments) may increase homeostatic momentum for sleep and increase deep sleep for recovery the next night. In this case, the higher the value of n in the n state, the deeper the sleep indication be. In this case, sleep onset latency (SOL) (20) may indicate a state of light sleep, which may indicate the state of the user within a certain first hour after falling asleep. A brief awakening level (21) may indicate a state of temporary awakening, i.e., light sleep, and slow wave sleep (SWS) level (22) may indicate a baseline for the stage of falling into deep sleep. The graph shows each sleep state, and as mentioned above, it can be set to mine virtual currency with different weight amounts according to each sleep state. At this time, mining virtual currency can be done universally.

FIG. 5 is a drawing of the same work of mining virtual currency according to the user's sleep state by one embodiment.

Looking specifically at FIG. 5, the server can classify the user's sleep state as the 1st˜5th stage sleep state (501) and set the mining weight differently depending on the classified sleep (502).

The server can determine the sleep state based on the user's physical condition. At this time, the sleep state can be classified into stage 1 sleep, stage 2 sleep, stage 3 sleep, stage 4 sleep, and stage 5 sleep based on EEG information, pulse rate information, body temperature information, pupil movement information, and breathing pattern information, as described below, and can be classified into stage 3, 4, or 6 or more stages. The server can set the mining amount of the virtual currency according to the sleep state.

The user's sleep state is classified into the first stage of sleep, the second stage of sleep, the third stage of sleep, the fourth stage of sleep and the fifth stage of sleep based on the brain wave information, pulse rate information, body temperature information, pupil movement information and breathing pattern information, and based on the user's physical state, the output of the virtual currency mining signal may be set differently according to the first stage of sleep, the second stage of sleep, the third stage of sleep, the fourth stage of sleep and the fifth stage of sleep. Pupil movement information during sleep can be obtained universally, and it can also be obtained through information stored in the user's device.

At this time, the user's sleep state is not limited to the above, but sleep can also be classified according to the following conditions. It is possible to use brain waveforms or recordings of breathing and sleep sounds, including heart rate, to recognize the weights of each section and to differentiate them accordingly and mine them in the form of proof of technology.

Stage 1˜5 sleep can also be classified according to the following criteria. Stage 1 may indicate REM sleep. The classification of the first stage of sleep is based on the user's pupil movement information, if the user's pupil moves more than once or more in a predetermined distance within the predetermined first hour, and the number of breaths of the user is less than a predetermined cycle, based on the user's breathing information, and the user's breathing sound is more than a predetermined decibel, the user's sleep state can be classified as the first stage sleep.

As an embodiment, the corresponding virtual currency mining weight (hereinafter referred to as the mining weight) may correspond to 1.5. At this time, the first stage of sleep is based on brain waves corresponding to gamma waves (30 Hz˜45 Hz). It is characterized by a fast EEG cycle and frequent eye movements, so it is possible to take advantage of the period of active brain activity and dreaming. At this time, the average heart rate can be about 60˜90 bpm, and although the heart rate increases due to active brain activity and eye movement, it may still be lower than the initial stage of sleep.

Stage 2 sleep may indicate NREM (non-REM) sleep. As an embodiment, the second stage sleep can be set to 1.0 with a mining weight, and the brain wave can be an alpha wave (8˜13 Hz). At this time, the section where muscle tension decreases and fast and small brain waves appear can be used as fast and irregular brain waves, and the average heart rate can be about 60˜100 bpm. In the early stages of sleep, the heart rate may be empirically faster, but it may be possible to take advantage of the intervals that are generally higher than normal sleep. The third sleep stage may represent NREM2 (Stage2) sleep, and the mining middle value may correspond to 1.3 as a daily embodiment. At this time, the brain wave can be by theta wave (4˜7 Hz), and it is a mixed form of rapid brain wave cycle (spindles) and delta brain wave cycle, and the brain wave becomes more complex, and the interval of starting to dream can be used as an intermediate stage without eye movement. At this time, the average heart rate: may be approximately 60˜100 bpm, and the interval in which the heart rate decreases slightly and stabilizes within the normal range can be utilized.

The fourth sleep stage may be the NREM3 (Stage 3) stage. As an embodiment, the mining weighting value may be 1.7, and the brain wave may be based on the delta wave (0.5˜0.7 Hz). At this time, it can be mainly used as the delta brain wave cycle, which further reduces muscle tension and allows the body to enter a deep sleep state, and the average heart rate can be about 40˜60 bpm. At this time, the heart rate is at its lowest and stable during the deep sleep phase. The heart rate is the lowest within the normal range, and the burnout zone can be utilized.

The fifth surface stage may be NREM4 (Stage 4), with a mining weight of 1.9 as a daily embodiment. Brain waves can be delta waves (0.5˜0.7 Hz), and they can be mainly used as a delta brain wave cycle, which further reduces muscle tension and allows the body to enter a deep sleep state. At this time, the average heart rate can be approximately 40˜60 bpm, and the heart rate is lowest and stable in the deep sleep phase. The heart rate can be used in the lowest range within the normal range.

The five levels may be weighted according to the total amount of sleep, or they may be weighted by the following: The more sleep you get, the more additional weight you get. At this time, it can be set to correspond to the total amount of mined weight sleep*1.0 when sleeping for less than 4 hours as the 1st hour day embodiment, the 2nd hour which is more than the 1st hour, the mining weighted total sleep amount*1.38 can be set to be added when sleeping less than 6 hours as a temporary example, the 3rd hour which is more than the 2nd hour, the mining weighted total amount of sleep*1.7 can be set to be added when sleeping less than 8 hours as a daily embodiment, and the 4th hour which is more than the 3rd hour, As an embodiment, if you sleep for more than 10 hours, you can set a weight corresponding to the mining weight total sleep amount*2.0. In addition, it can be set to increase the weight of sleep stages for a specific period.

As an embodiment, the mining weight can be set to*3.0 in the REM sleep section. At this time, the embodiments mentioned above are only and not limited to one embodiment. In addition, after declaring a specific week or month sleep target time, it can be set to increase the mining weight when the goal is achieved. In addition, when you hold a specific NFT (Non-Fungible Token), the mining value can be set to increase.

In addition, it can be set to increase the mining weight when beneficial sounds are produced or provided to the user during sleep. In this case, the beneficial sound may represent a predetermined sound.

At this time, the amount of cryptocurrency mining can be determined by the following mathematical [Equation 1].

$${α}_{value} = \sqrt{{\sum }_{a - 1}^{b} {( {x}_{a - sleepv} \times {y}_{a - sleepw} \times {TIME}_{a - sleep} )}^{2}} + {Q}_{δ} \qquad [ Equation 1 ]$$

The & value may represent the amount of virtual currency mining. The b may represent the number of sleep states including the first sleep, the second sleep state, the third sleep state, the fourth sleep state, and the fifth sleep state, and may represent five in one embodiment. The Xₐ₋ₛₑₑₚᵥ represents the sleep reward corresponding to the ath sleep state, which can represent the virtual currency reward value obtained through sleep. At this time, the sleep reward is the basic reward given to the user, and it can be set to be differentiated according to the situation according to the period and region, and it may be a fixed value. One embodiment may be fixed at 1.

The y_{a-sleepw} represents the weight of the sleep stages and can represent the weight corresponding to the ath sleep state of the above embodiment. As mentioned above, different weights can be set according to the sleep state, and the mining revenue of the period can be weighted and averaged. For example, REM in the first sleep state has a weight of 1.5, NREM1 in the second sleep state has a weight of 1.0, NREM2 in the third sleep state has a weight of 1.3, the fourth sleep state, NREM3, can be set to a weight of 1.7, and the fifth sleep state, NREM4=weight, to 1.9. The corresponding numerical equation 1 can be converted to square root using a root function (sqrt). The Q_{δ} may represent a random weight between the respiratory rate 1 and 24 based on the breathing pattern information. At this time, it is possible to differentiate according to the average breathing rate set in advance through the user information to add virtual currency mining profits. The TIMEₐ₋ₛₗₑₑₚ can represent the time corresponding to the sleep state a.

The Q_{δ} can be derived by the following mathematical [Equation 2].

$${Q}_{δ} = {T}_{breath} \times {y}_{a - sleepw} \qquad [ Equation 2 ]$$

The T_{breath} may indicate the number of breaths during a predetermined period. At this time, the predetermined time may be determined based on the user's usual respiratory cycle, and the respiratory cycle of the user's usual sleep state. The pre-specified time may be set so that the user's respiratory rate is 24 or less for a predetermined time, based on the user's usual respiratory cycle and the respiratory cycle of the user's usual sleep state. At this time, the predetermined time can be set differently for each sleep stage. As an embodiment, the time can be set to lengthen or shorten from steps 1 to 5. yₐ₋ₛₗₑₑₚ may represent the weight corresponding to the a-th sleep state.

You can use this formula to adjust your mining activities by focusing on the desired stage and to vary your expected returns by weighing your breathing rate. As mentioned above, it is possible to obtain individually differentiated mining values through combined weights, root functions, and other variables.

At this time, when the amount of cryptocurrency mining is determined, the server can output a signal to mine the amount of cryptocurrency mining. The amount of cryptocurrency mining is not limited to this, but it can be proportional to the number of pupil movement information, and the amount can be set differently depending on body temperature, and the amount can be set differently depending on the brain wave state or health condition.

For example, it can be set to mine more amounts, if the EEG corresponds to a certain frequency, if the user's body is judged to be healthier, and if the user's body temperature is within a certain body temperature range.

In addition, the amount of cryptocurrency mining can be determined by comparing the user's sleep pattern with the user's usual sleep pattern.

After obtaining the user's sleep pattern for a predetermined period and determining the sleep pattern as an average or a user-specified value (e.g., the average REM sleep time of the user and the degree of respiration during REM sleep, body temperature, pupil movement, heart rate, etc.), and then comparing it with this, if the user sleeps more deeply than usual, the amount of mining can be increased. If you sleep more lightly than usual, you can reduce the amount of mining. At this time, if you sleep more deeply or lightly than usual, you can set the amount of mining to change more or less depending on the difference. At this time, it is not limited to the above embodiment, but it is possible to reduce the amount of mining if you sleep deeply in half. In addition, it can be set to be mined only at a specific time set by the user, and it can be linked to the user's schedule information to reduce, decrease, or not mine the amount of mining according to the predetermined date, event schedule, or specific customization by the user. In addition, it can be set to adjust the mining amount based on a specific event or SNS information and news information obtained from an external server to reflect trends. For example, if the index of the stock market rises or falls above a certain threshold, or if information about a particular content is posted by news information a certain number of times or more, the amount of mining can be reduced or decreased.

In an electronic device comprising at least one processor, at least one processor may obtain physical activity information from the user's terminal, determine the physical condition of the user based on the physical activity information, and output the virtual currency mining signal based on the physical condition of the user. In addition, communication interfaces, memory, etc., and other commonly used devices may be included.

The methods according to the present invention may be implemented in the form of programmatic instructions that may be performed by various computer means and recorded on a computer-readable medium. Computer-readable media may contain program commands, data files, data structures, etc., alone or in combination. Programmed commands recorded on a computer-readable medium may be designed and constructed specifically for the present invention or may be available for use by a computer software practitioner.

Examples of machine-readable media may include hardware devices that are specifically configured to store and perform program instructions, such as ROM, RAM, flash memory, and so on. Examples of program instructions may include machine code, such as those generated by compilers, as well as high-level language code that can be executed by computers, such as interpreters. The aforementioned hardware device may be configured to operate as at least one software module to operate the present invention, and vice versa.

In addition, the aforementioned method or apparatus may be implemented in combination with all or part of its configuration or function, or it may be implemented separately.

Although the above has been described concerning the preferred embodiment of the present invention, it can be understood that a skilled craftsman in the field of technology can modify and modify the present invention in various ways within the scope of the idea and scope of the present invention described in the scope of the patent claims below.

The methods according to the present invention may be implemented in the form of programmatic instructions that may be performed by various computer means and recorded on a computer-readable medium. Computer-readable media can contain program instructions, data files, data structures, etc., alone or in combination. Programmed commands recorded on a computer-readable medium may be designed and constructed specifically for the present invention or may be available for use by a computer software practitioner.

Examples of machine-readable media may include hardware devices that are specifically configured to store and perform program instructions, such as ROM, RAM, flash memory, and so on. Examples of program instructions may include machine code, such as those generated by compilers, as well as high-level language code that can be executed by computers, such as interpreters. The aforementioned hardware device may be configured to operate as at least one software module to operate the present invention, and vice versa.

In addition, the aforementioned method or apparatus may be implemented in combination with all or part of its configuration or function, or it may be implemented separately.

Although the above has been described concerning the preferred embodiment of the present invention, it can be understood that a skilled craftsman in the field of technology can modify and modify the present invention in various ways within the scope of the idea and scope of the present invention described in the scope of the patent claims below.

1. A method for mining virtual currency according to the physical state of a user, performed by a server, comprising:
acquiring physical activity information from a user terminal;
determining the physical state of the user based on the physical activity information;
outputting a signal to mine virtual currency based on the physical state of the user.

2. The method of claim 1, wherein the physical activity information includes at least one of the user's EEG information, pulse rate information, body temperature information, pupil movement information, and breathing pattern information.

3. The method of claim 2, further comprising:
determining the sleep state based on the physical state of the user;
outputting a signal to mine virtual currency based on the physical state of the user, wherein the step of outputting the signal includes:
setting the mining weight of the virtual currency differently according to the sleep state.

4. The method of claim 3, wherein the user's sleep state is classified into N stages of sleep based on at least one of the EEG information, pulse rate information, body temperature information, pupil movement information, and breathing pattern information, and the step of outputting the signal to mine virtual currency based on the physical state of the user includes:
setting the mining weight of the virtual currency differently according to the N stages of sleep,
wherein N represents an integer of 1 or more.

5. The method of claim 4, wherein the N stages of sleep include a first stage of sleep, and the step of classifying the user's sleep state as the first stage of sleep includes:
classifying the user's sleep state as the first stage of sleep based on the user's pupil movement information if the user's pupil moves more than a predetermined first distance within a predetermined first time, and based on the user's breathing information if the number of breaths of the user is less than a predetermined cycle and the user's breathing sound is more than a predetermined decibel.

6. An electronic device comprising at least one processor, wherein the at least one processor:
acquires physical activity information from a user terminal;
determines the physical state of the user based on the physical activity information;
outputs a signal to mine virtual currency based on the physical state of the user.
